(19) United States
(12) Patent Application Publication
(10) Pub. No.: US 2024/0415916 A1
(43) Pub. Date: Dec. 19, 2024

(54) COMPOSITION OF A SWEET CORN SILK EXTRACT FOR MEDICINAL USE AND A METHOD THEREOF

(71) Applicant: Botanic Healthcare Pvt. Ltd., Hyderabad (IN)

(72) Inventors: Gaurav Soni, Hyderabad (IN); Madhu Krishnamani, Hyderabad (IN); Hebbani Nagarajappa Shivaprasad, Hyderabad (IN); Sharanabasappa Gundappa Kinni, Hyderabad (IN); Garaga Machiraju, Hyderabad (IN); Arnab Chatterjee, Hyderabad (IN); Divanji Manohar Shriranga, Hyderabad (IN); Sravani Thimmannagari, Hyderabad (IN); Srivalli Susmitha Ghatti, Hyderabad (IN)

(21) Appl. No.: 18/746,383

(22) Filed: Jun. 18, 2024

(30) Foreign Application Priority Data
Jun. 19, 2023 (IN) 202341041427

(51) Int. Cl. A61K 36/899; A61K 31/05; A61K 31/375; A61K 31/7048; A61K 33/00

(52) U.S. Cl. CPC A61K 36/899; A61K 31/05; A61K 31/375; A61K 31/7048; A61K 33/00; A61K 2236/15; A61K 2236/333; A61K 2236/51

(57) ABSTRACT

A composition 100 of a sweet corn silk extract 102 for a medicinal use is provided. The composition includes 0.25-0.50 percent weight of Maysin 104, 2.0-3.0 percent weight of natural potassium 106, 7.5-9.0 percent weight of total polyphenols 108, and 1.0-2.0 percent weight of vitamin C 110.

This Application claims priority from a complete patent application filed in India having Patent Application No. 202341041427, filed on 19th day of June 2023, and titled “A COMPOSITION OF A SWEET CORN SILK EXTRACT FOR MEDICINAL USE AND A METHOD THEREOF”.

### FIELD OF INVENTION

Embodiments of the present disclosure relate to the technical field of extraction of plant, and more particularly to a composition of a sweet corn silk extract for medicinal use and a method thereof.

### BACKGROUND

A variety of herbal extracts are having medicinal effects such as solutions to anti-inflammatories, solutions to obesity, and the like. Specifically, obesity increases the risk of several debilitating, and deadly diseases, including diabetes, heart disease, and some cancers. Solutions to such diseases need to be addressed in a such a way that there may not be any other side effects on the body. For extraction, various food elements may be used such as corn, corn silk, and the like.

Various compositions of herbal extract include Maysin, which is used for treating Alzheimer's disease, obesity, and the like. The current, existing, herbal extracts do not improve the tyrosinase inhibitory effect and significantly increase the content of Maysin, polyphenol, and flavonoid through enzyme treatment. The existing extraction process extracts with only Maysin or flavones which does not provide enhancing effects on the medicine used in obesity.

Hence, there is a need for a composition of a sweet corn silk extract for medicinal use and a method thereof to address the aforementioned issue(s).

### OBJECTIVE OF THE INVENTION

An objective of the invention is to provide a composition of sweet corn silk extract from a process for standardized sweet corn silk extract for medicinal use.

Another objective of the present invention is to provide a composition to be used in anti-obesity applications.

### BRIEF DESCRIPTION

In accordance with an embodiment of the present disclosure, a composition of a sweet corn silk extract for medicinal use is provided. The composition includes Maysin, natural potassium, total polyphenols, and vitamin C. The composition includes 0.25-0.50 percent weight of Maysin, 2.0-3.0 percent weight of natural potassium, 7.5-9.0 percent weight of total polyphenols, and 1.0-2.0 percent weight of vitamin C.

In accordance with another embodiment of the present disclosure, a method for extraction of sweet corn silk and compositions thereof is provided. The method includes cleaning, of sweet corn silk using demineralized water. The method also includes drying a cleansed sweet corn silk at a pre-defined temperature. Further, the method includes pulverizing the dried sweet corn silk by a milling tool to convert the dried sweet corn silk into a liquid form. Furthermore, the method includes extracting the converted liquid form of the dried sweet corn silk over a plurality of pre-defined cycles and at a pre-defined time interval. Furthermore, the method includes filtering, the extracted liquid sweet corn silk by using celite to obtain a filtrate. Furthermore, the method includes concentrating, the filtrate by distillation under a vacuum to a total dissolved solid content to obtain a concentrated sweet corn silk extract. Furthermore, the method includes spray-drying, concentrated sweet corn silk extract to get a laminar flowing hygroscopic powder.

To clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a composition of a sweet corn silk extract for medicinal use in accordance with an embodiment of the present disclosure; and

FIG. 2 is a flow chart representing the steps involved in a method of extraction of sweet corn silk and compositions thereof in accordance with an embodiment of the present disclosure.

Further, those skilled in the art will appreciate that elements in the figures are illustrated for simplicity and may not have necessarily been drawn to scale. Furthermore, the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a composition of a sweet corn silk extract for medicinal use. The composition includes Maysin, natural potassium, total polyphenols, and vitamin C. Further, the composition includes 0.25-0.50 percent weight of Maysin, 2.0-3.0 percent weight of natural potassium, 7.5-9.0 percent weight of total polyphenols, and 1.0-2.0 percent weight of vitamin C.

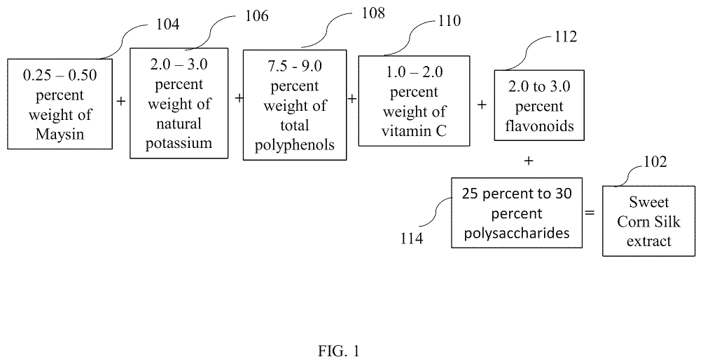

FIG. 1 is a block diagram representation of a composition (100) of a sweet corn silk extract for medicinal use in accordance with an embodiment of the present disclosure. The composition 100 of a sweet corn silk extract 102 for a medicinal use includes 0.25-0.50 percent weight of Maysin 104. In one embodiment, the molecular formula of Maysin 104 is C27H28O14, and the molecular weight of the Maysin 104 is 576.

The composition 100 also includes 2.0-3.0 percent weight of natural potassium 106. Further, the composition 100 also includes 7.5-9.0 percent weight of total polyphenols 108. Furthermore, the composition 100 includes a 1.0-2.0 percent weight of vitamin C 110. In one embodiment, the composition 100 includes total flavonoid 112 content as Rutin, wherein the total flavonoid 112 content as Rutin includes a concentration of 2.5-3.5 percent weight. The composition 100 includes 25-30 percent weight of total polysaccharides 114. In one embodiment, the composition 100 includes 2 to 5 percent weight of moisture 116 content.

In one embodiment, the composition 100 includes the Maysin 104, not less than 0.25 percent weight, the Vitamin C 110 not less than 0.5 percent weight, the natural potassium 106 not less than 2.0 percent weight, total flavonoids 112 not less than 2.5 percent weight (as Rutin), total polyphenols 108 not less than 7.5 percent weight (as gallic acid), the polysaccharides 114 not less than 25 percent weight, moisture 116 not more than 5.0 percent weight.

In one embodiment, the composition described in paragraph [0021] may be the preferred embodiment for anti-obesity applications.

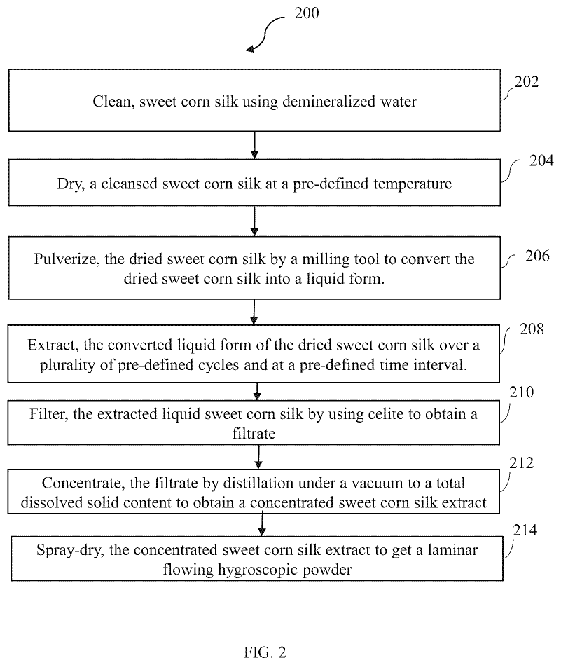

FIG. 2 is a flow chart representing the steps involved in for extraction of sweet corn silk and compositions thereof in accordance with an embodiment of the present disclosure. The method includes cleaning of sweet corn silk using demineralized water in step 202.

The method 200 also includes drying a cleansed sweet corn silk at a pre-defined temperature in step 204. In one embodiment, the predefined temperature is 40 to 45 degrees Celsius.

Further, the method 200 includes pulverizing the dried sweet corn silk by a milling tool to convert the dried sweet corn silk into a liquid form in step 206.

Furthermore, the method 200 includes extracting the converted liquid form of the dried sweet corn silk over a plurality of pre-defined cycles and at a pre-defined time interval in step 208. The method also includes filtering through celite, concentrated by distillation under vacuum to the total dissolved solid content of 40 percent. In one embodiment, the concentrate dried with an inlet temperature of 160 degree Celsius and outlet temperature at 100 degrees Celsius to get a free-flowing hygroscopic sweet corn silk extract liquid with 25 percent yield from the raw material.

Furthermore, the method 200 includes filtering, the extracted liquid sweet corn silk by using celite to obtain a filtrate in step 210.

Furthermore, the method 200 includes extracting sweet corn silk liquid using five volumes of 30 percent ethanol in water at 60 degrees Celsius. The method also includes extracting the sweet corn silk liquid from a sweet corn silk using five volumes of 30 percent ethanol in water at 60 degree Celsius for 4 hr each of three cycles.

Furthermore, the method 200 includes concentrating, the filtrate by distillation under a vacuum to a total dissolved solid content to obtain a concentrated sweet corn silk extract in step 212. The method also includes concentrating, the filtrate by distillation under vacuum to a total dissolved solid content of 40 percent.

Furthermore, the method 200 includes spray-drying, the concentrated sweet corn silk extract to get a laminar flowing hygroscopic powder in step 214. The method also includes spray-drying, the concentrated sweet corn silk extract with an inlet temperature at 160 degrees Celsius and an outlet temperature of 100 degrees Celsius. Various embodiments of the composition of the sweet corn silk extract are used for medicinal purposes. The composition disclosed in the present disclosure provides a composition of sweet corn silk extract from a process for standardized sweet corn silk extract for medicinal use. The composition in the present disclosure provides a composition anti-obesity application. The method of extraction disclosed in the present disclosure uses a green process for the extraction of sweet corn silk and enrichment of phenolic acids and Flavonoids.

The present disclosure discloses the raw material used for extraction hence the user may trust the source and its effectiveness. The composition disclosed in the present disclosure provides an effective and safe solution to the obesity problem. The composition made by using the method described in the present disclosure is used in proportional content of Maysin, to avoid harmful effects of consumption of excess Maysin.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. A composition of a sweet corn silk extract for a medicinal use comprises:
characterized in that:
0.25-0.50 percent weight of Maysin;
2.0-3.0 percent weight of natural potassium;
7.5-9.0 percent weight of total polyphenols; and
1.0-2.0 percent weight of vitamin C.

2. The composition as claimed in claim 1, wherein the molecular formula of Maysin is C27H28O14, and the molecular weight of the Maysin is 576.

3. The composition as claimed in claim 1, comprises total flavonoid content as Rutin, wherein the total flavonoid content as Rutin comprises a concentration 2.5-3.5 percent weight.

4. The composition as claimed in claim 1, comprises 25-30 percent weight of total polysaccharides.

5. The composition as claimed in claim 1, comprises 2 to 5 percent weight of moisture content.

6. A method for extraction of sweet corn silk and compositions thereof, wherein the method comprises:
characterized in that:
cleaning, of sweet corn silk using demineralized water;
drying a cleansed sweet corn silk at a pre-defined temperature;
pulverizing the dried sweet corn silk by a milling tool to convert the dried sweet corn silk into a liquid form;
extracting, the converted liquid form of the dried sweet corn silk over a plurality of pre-defined cycles and at a pre-defined time interval;
filtering, the extracted liquid sweet corn silk by using celite and obtaining a filtrate;
concentrating, the filtrate by distillation under a vacuum to a total dissolved solid content to obtain a concentrated sweet corn silk extract; and
spray-drying, the concentrated sweet corn silk extract to get a laminar flowing hygroscopic powder.

7. The method as claimed in claim 6, wherein the predefined temperature is 40 to 45 degrees Celsius.

8. The method as claimed in claim 6, comprises extracting sweet corn silk liquid using five volumes of 30 percent ethanol in water at 60 degrees Celsius.

9. The method as claimed in claim 6, wherein the filtrate is concentrated by distillation under vacuum to a total dissolved solid content of 40 percent.

10. The method as claimed in claim 6, wherein the concentrated sweet corn silk extracts spray-dried with an inlet temperature at 160 degrees Celsius and an outlet temperature of 100 degrees Celsius.
